(19) United States
(12) Patent Application Publication
(10) Pub. No.: US 2010/0004640 A1
(43) Pub. Date: Jan. 7, 2010

(54) Cancer Treatment Using Lasers

(75) Inventors: Densen Cao, Sandy, UT (US); Steve D. Jensen, South Jordan, UT (US)

Correspondence Address: GEOFFREY E. DOBBIN, PATENT ATTORNEY, 4278 SOUTH 6220 WEST, WEST VALLEY CITY, UT, 84128-6501, US

(73) Assignee: CAO GROUP, INC., West Jordan, UT (US)

(21) Appl. No.: 12/555,692

(22) Filed: Sep. 8, 2009

Related U.S. Application Data
(63) Continuation-in-part of application No. 11/787,899, filed on Apr. 18, 2007, now abandoned.
Application No. 11/787,899 is a continuation-in-part of application No. 11/210,276, filed on Aug. 23, 2005, now abandoned.
Application No. 11/210,276 is a continuation-in-part of application No. 11/423,424, filed on Jun. 9, 2006, now abandoned.

(51) Int. Cl. A61B 18/20

(52) U.S. Cl. 606/2

(57) ABSTRACT

A method and apparatus for destroying cancerous cells or tumors includes placing fiber needles into the human body adjacent cancerous cells or tumors that have been biologically stained and exposing the cells or tumors to low-energy laser energy light emitted through the fiber needles so that the laser energy destroys the cancer cells or tumors through carbonization and/or vaporization without destruction of surrounding healthy tissue. The stain is specifically selected to have an absorption efficiency of greater than 90% for energy emitted by a given laser such that it greatly enhances absorption of the laser energy over surrounding unstained tissue. Appropriate stain and laser selection can allow treatment through an intact column of living tissue as laser energy to which living tissue is transparent may be used in combination with a stain that makes targeted tissue opaque to that energy.

### RELATED APPLICATIONS

The present application is a continuation-in-part of U.S. patent application Ser. No. 11/787,899, filed Apr. 18, 2007, which is in turn a continuation-in-part of U.S. patent application Ser. No. 11/210,276, filed Aug. 23, 2005 and U.S. patent application Ser. No. 11/423,424, filed Jun. 9, 2006.

### TECHNICAL FIELD OF THE INVENTION

The present invention relates to treatment of cancers and, more particularly, to equipment and methods used in the treatment of cancerous tumors using lasers.

### BACKGROUND OF THE INVENTION

Known treatments for cancer include radiation, surgery, drugs, thermal ablation, photodynamic therapy, and other means. While these methods exhibit various degrees of success, the methods also exhibit various undesirable side effects and, further, prove ineffective in destroying cancerous tumors under certain circumstances. One area of research currently receiving great interest concerns the use of lasers. In photodynamic therapy (PDT), for example, laser light of a specific wavelength may be used to activate a photosensitizing agent previously introduced into the blood stream. Interaction of the laser light with the agent produces an active form of oxygen that destroys nearby cancer cells. Drawbacks to this method include, however, the need for the patient to avoid direct sunlight or bright indoor light for several weeks following treatment and ineffectiveness of active agents in PDT methods to completely destroy tumors. Side effects can also include burns, swelling, pain and scarring of nearby tissue.

Laser-induced interstitial thermotherapy (LITT) is another laser-based clinical tool for treating various malignancies. With LITT, bare fibers or diffusing applicators are punctured into the pathological volume to distribute the laser energy within the region of interest, raising the temperature of cancerous cells and destroying them. A concern for both PDT and LITT is proper focusing of the laser light to the precise area of the tumor. If the laser is too powerful, for example, cell tissue adjacent or underlying the cancerous tumor can become damaged or destroyed, leading to adverse side effects.

The use of lasers for cancer treatment presents other concerns. One particular concern relates to the generally precise tuning of laser energy output and the significant range of absorption efficiencies that accompany various different body tissues. More specifically, since a specific type of laser generally provides an output that is tuned to a narrow wavelength range, it is rare that the range will correspond to the most efficiently absorbed wavelength of a particular subjected tissue. This drawback follows two main observations. The first observation is that different regions or layers of biological tissue that may require treatment in the same procedure will exhibit different absorption efficiencies—e.g. one region may absorb laser energy more efficiently than another—thus necessitating a laser that will treat a variety of regions or layers somewhat efficiently on average, but never precisely. One result of this observation is that tissues exhibiting relatively low absorption efficiency are subject to being treated with a laser having a higher energy output than necessary, which may lead to over-ablation or penetration into underlying regions or layers to cause damage in healthy tissues. Secondly, different people will have different shades of tissue, in particular skin tone, when compared to others and on various parts of their own bodies (e.g. moles). A single laser operating at a specific output frequency will generally not be tuned to the variety of optimal absorption efficiencies that the variety of tissues exhibit between persons or between different tissues on the same person. Indeed, even if a single laser were tuned to operate at a frequency consistent with the optimal absorption efficiency of a particular patient's tissue under treatment, the laser's effectiveness would likely change at the instant a procedure (e.g. a mole removal) was complete and before the laser could be shut down. In either case—i.e., inter person or intra person treatment—the imprecise tuning of the laser to the tissue causes some degree of over-penetration. Over-penetration is the exposure and potential destruction of a column of tissue underlying the targeted tissue to unabsorbed radiant energy as it spills into deeper biological layers. Over-penetration typically causes a blistering effect as fluid released from the unwanted destruction of tissues is expressed through the wound caused by the procedure.

The present invention reduces the chance that cell tissue adjacent or underlying the cancerous tumor is damaged or destroyed while cancerous tissues are being treated. The present invention accomplishes this objective through use of laser light that is tuned to interact with dye substances injected directly or systemically and/or painted onto the cancerous tumor. The precise tuning of the laser light with the dye increases the efficiency or absorption rate at which laser energy is absorbed by the tissue comprising the cancerous tumor, thereby allowing the use of relatively low energy lasers and reducing the chance that energy from the laser is permitted to reach and damage or destroy outer lying healthy tissue. The present invention also comprises a method of staining a given biological substrate for attunement to a given laser source, rather than the other way around as is practiced in the prior art. When employed with the methods disclosed herein, suitable lasers can be used on any biological substrate regardless of the output wavelengths produced. The use of a stain also concentrates the laser's radiant energy in the stained tissues, lessening over-penetration by forcing precise attunement of the tissues to the laser output. In addition, a substance that is opaque to a particular radiant energy can be applied around the stained treatment area to protect against incidental or accidental exposure of laterally located tissues to harmful radiant energy during treatment. Given the cost advantage of producing and purchasing a stain over a laser, the method of the present invention represents an extremely cost beneficial advancement in the art.

For example, the absorption rate of laser energy by tissue depends on the wavelength of the laser light, and the optimal wavelength will depend on the particular cell tissue being treated. Thus, the amount of laser energy required to destroy a cancerous tumor will vary depending on the particular tissue being treated. This leads to a situation where coherent energy from a laser operating at a particular wavelength will be efficient at destroying some tissues but not others. Further, a tissue having a relatively high absorption rate of laser energy for a specific wavelength will be destroyed over a shallow tissue depth than one having a relatively low absorption rate. Conversely, a tissue having a relatively low absorption rate will require a higher incident flux of energy (or the same flux incident over longer periods of time) for the same amount of destruction to occur since the energy is being distributed throughout a deeper column of tissue. The variation in the absorption rate of incident energy can lead to over-penetration. In other words, if an energy flux incident on a tumor having a certain depth is not completely absorbed by the tumor over the tumor depth, the incident flux may over-penetrate into one or more underlying layers of tissue. This situation can be critical, especially if a surgery would be considered a failure if laser energy penetrates beyond the treatment zone and damages delicate tissues that surrounds or underlies the zone.

The present invention avoids the problem of over-penetration through use of laser light in conjunction with a biological dye to treat cancerous tumors. Biological dyes can be selected to “match” specific wavelengths of laser energy, thereby helping to contain the laser energy in a localized zone. This occurs because certain dyes increase the absorption rate of laser energy of a specific wavelength. Since certain dyes absorb light much more efficiently than tissues, one can selectively “stain” a tumor of interest and destroy only that selected tissue or tumor, minimizing damage to un-stained tissue. Thus, one can increase the absorption rate of laser energy in a localized tissue area through proper selection of the dye. Increased absorption efficiency allows use of less powerful lasers, thereby reducing the chance that surrounding tissue will be damaged or destroyed—healthy cells adjacent the tumor and not containing the dye sustain minimal damage. In addition, because specific dyes can also be matched to specific coherent laser energy sources, the dye also provides a means to control “over-penetration.”

This procedure allows for “low-energy destruction” of cancerous tumors, which provides a much safer means to perform tumor or tissue treatments. The described methods can employ relative low laser energy settings and are safer, since the low-energy laser will produce far less damage or destruction of healthy surrounding tissue through accidental or incidental exposure of laser energy. By the same reasoning, low-energy tissue or tumor destruction also minimizes the risk of over-penetration of unabsorbed light energy traveling beyond the intended zone of penetration.

### SUMMARY OF THE INVENTION

A method for treating a cancerous tumor or cells using a laser system matched with a dye, stain, or pigment is invented. A region within a body that contains a cancer tumor or cells is located using conventional steps such as laser scanning, magnetic resonance imaging, x-ray imaging, or CT scans. A dye is then injected directly or systemically and/or painted into a tumor, tumors, or tissues. Stained tumor, tumors, or tissues are exposed to a radiant energy source which having a wavelength closely matching the absorption characteristics of the dye. Emission of laser light from the laser system is applied, to the tumor or cells, and continues for a medically effective duration in order to destroy at least a portion of the tumor or cells by either carbonizing or vaporizing the tumor or cells.

An embodiment of the invention includes use of a plurality of fibers, through which the laser light may be emitted. A further embodiment comprises use of a biological dye selected from the group consisting of indocyanine green, carbon black, FD&C Blue #2, and nigrosin, FD&C black shade, FD&C blue #1, methylene blue, FD&C blue #2, malachite green, D&C green #8, D&C green #6, D&C green #5, ethyl violet, methyl violet, FD&C green #3, FD&C red #3, FD&C red #40, D&C yellow #8, D&C yellow #10, D&C yellow # 11, FD&C yellow #5, FD&C yellow #6, neutral red, safranine 0, FD&C carmine, rhodamine G, napthol blue black, D&C orange #4, thymol blue, aurarnine 0, D&C red #22, D&C red #6, xylenol blue, chrysoidine Y, D&C red #4, sudan black, B, D&C violet #2, D&C red #33, cresol red, fluorescein, fluorescein isothiocyanate, bromophenol red, D&C red #28, D&C red #17, amaranth, methyl salicylate, eosin Y, Lucifer yellow, thymol, and dibutyl phthalate. A further embodiment comprises selection of the dye wherein the wavelength of the laser light is absorbed by the tumor or cells containing the dye and wherein the laser light passes harmlessly through healthy cells that surround the tumor or cells.

The more important features of the invention have been outlined in order that the more detailed description that follows may be better understood and in order that the present contribution to the art may better be appreciated. Additional features of the invention will be described hereinafter and will form the subject matter of the claims that follow. Many objects of this invention will appear from the following description and appended claims, reference being made to the accompanying drawings forming a part of this specification wherein like reference characters designate corresponding parts in the several views. Before explaining at least one embodiment of the invention in detail, it is to be understood that the invention is not limited in its application to the details of construction and the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

Those skilled in the art will, therefore, appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

FIG. 1 depicts a laser system of the present invention that can be used for the treatment of cancerous tumors.

FIG. 2 depicts a fiber needle of the present invention that can be used to deliver laser energy to tissue cells of a cancerous tumor.

FIG. 3 depicts a plurality of fiber needles of the present invention positioned to concentrate from multiple directions laser energy to tissue cells of a cancerous tumor.

FIG. 4 depicts a flow chart of the present invention showing a sequence of steps used in applying laser energy to a cancerous tumor.

FIG. 5 is a partial sectional view depicting an alternate embodiment of the invention.

### DEFINITIONS USED IN THIS SPECIFICATION

As used in this Application, the term “carbonize” shall mean “to apply energy to organic matter until it turns into carbon and/or oxides resulting from combustion.” The term “vaporize” shall mean “to convert an object or compound into vapor.” Both processes will occur in a tumor or tissue subjected to the methods described in this Application and this Application specifically and exclusively deals with the destruction of undesired tissue through these processes. Accordingly, as used in this Application, the term “destroy”, then, shall mean “destroy through carbonization and/or vaporization. This definition shall be to the exclusion of any other methods of destruction. These are the definitions used throughout this entire Specification. As used herein, the term “stain” shall be understood to include all such dyes, pigments, stains, and any compound or solution utilizing such dye, pigment or stain as an ingredient in its combined whole. The use of the term “stain” is to be understood to include such “stains” that include a pigment or dye as its only ingredient.

### DETAILED DESCRIPTION OF THE PREFERRED EMBODIMENTS OF THE INVENTION

This invention concerns use of lasers in conjunction with dyes, stains or pigments to carbonize and/or vaporize tumors or tissues when identified and located. Currently used methods for identification of tumors include laser scanning, magnetic resonance imaging (MRI), x-ray imaging, CT scans, and other means. Following identification and location of tumor cells in the body, a dye, stain or pigment is attached to the identified tumor cells through injection or special agent. For example, certain stains by themselves or when combined with tumor seeking compounds can be injected systemically into the bloodstream, with the stain accumulating more efficiently in tumors than in healthy tissues. The accumulated stain is then imaged using X-ray, MRI or ultrasound devices or the like. Once located, the tumor is destroyed using a radiant energy delivering device—e.g., a fiber optic device. One benefit of this approach is the stain serves as both the imaging and sensitivity stain and, further, the only device requiring delivery to the tumor site is the radiant energy delivering device.

In other embodiments, an imaging chemical is systemically injected into the bloodstream, with the imaging chemical accumulating at a tumor more efficiently than in healthy tissues. The tumor is then identified using conventional imaging techniques. Identification of the tumor location is followed by systemic injection of a stain into the bloodstream, with the stain then attaching itself to the imaging chemical accumulated in or at the tumor. The tumor is then destroyed using a radiant energy delivering device. In yet other embodiments, an imaging chemical is systemically injected into the bloodstream, with the imaging chemical accumulating in or at a tumor more efficiently than in healthy tissues. Location of the tumor is then identified using conventional imaging techniques. A stain is then delivered to the tumor by mechanical means—e.g., a syringe—followed by destruction of the tumor through carbonization and/or vaporization using a radiant energy delivering device.

Laser energy using a fiber needle or fiber is directly delivered to the tumor cells which are already stained. Delivery of laser energy to the tumor cells can be accomplished using a single needle or a plurality of needles depending on the size of tumor. Multiple fiber needles can be inserted inside the body from multiple directions so that the cancer tumor can be covered or surrounded by laser energy completely. Such fiber needles generally include a reflective coating such that light is emitted only through an end or tip of the needle. A further embodiment includes a fiber needle not having a reflective coating such that light escapes along the entire fiber, thereby allowing a multi-directional treatment device. Regardless of the specific needle design, the laser is activated for a predetermined period of time. The tumor containing the stain will absorb the laser energy at a higher rate than surrounding tissue and be destroyed through carbonization, while surrounding tissue will remain mostly unaffected by the laser. Various details of the foregoing are disclosed in co-pending and commonly-owned U.S. patent application Ser. No. 11/210,276, entitled “Cancer Treatment Using Laser” and Ser. No. 11/423,424, entitled “Method of Marking Biological Tissues for Enhanced Destruction by Applied Radiant Energy,” the disclosure from both of which are incorporated herein in their entireties.

Lasers typically used to destroy tumors include solid state lasers, gas lasers, semiconductor lasers, and others. Typical wavelengths of electromagnetic radiation used in cancer treatments are from about 200 nm to about 8000 nm. Wavelengths outside this range may also be used. Typical power levels range from about 0.1 W to about 30 W, although greater or lesser power levels may be used in some circumstances. Typical treatment times for exposing cancerous cells to laser energy range from less than about 1 second to greater than about 1 hour, although longer or shorter times may be used. The laser energy applied to the tumor cells may also be modulated. Laser energy may be applied to tumor cells by continuous wave (constant level), pulsing (or/off), ramping (from low to high energy levels, or from high to low energy levels), or other waveforms (such as sine wave, square wave, triangular wave, etc.). Modulation of laser energy may be achieved by modulating energy to the laser light source or by blocking or reducing light output from the laser light source according to a desired modulation pattern.

Stains for use with the present invention include those stains having the ability to absorb laser energy at efficiencies higher than physiological tissues. As examples, the stain could be indocyanine green, carbon black, FD&C Blue #2, nigrosin or others. Further exemplar dyes, stains or pigments that are satisfactory in this regard include, but are not limited to: FD&C black shade, FD&C blue #1, methylene blue, FD&C blue #2, malachite green, D&C green #8, D&C green #6, D&C green #5, ethyl violet, methyl violet, FD&C green #3, FD&C red #3, FD&C red #40, D&C yellow #8, D&C yellow #10, D&C yellow #11, FD&C yellow #5, FD&C yellow #6, neutral red, safranine 0, FD&C carmine, rhodamine G, napthol blue black, D&C orange #4, thymol blue, auramine 0, D&C red #22, D&C red #6, xylenol blue, chrysoidine Y, D&C red #4, sudan black, B, D&C violet #2, D&C red #33, cresol red, fluorescein, fluorescein isothiocyanate, bromophenol red, bromophenol blue, D&C red #28, D&C red #17, amaranth, methyl salicylate, eosin Y, lucifer yellow, thymol, dibutyl phthalate, toluidine blue, and the like. The dye, stain or pigment may be applied by a pen, a brush, spraying, a fibrous pellet, a syringe tip, fiber syringe tip, small plastic tube, or otherwise. If desired, an opaque substance may be used to protect tissues, which are not to be cut or destroyed. Opaque substances could include titanium dioxide, zinc oxide, calcium carbonate, or otherwise.

The present invention represents a departure from the prior art in that the method of the present invention dictates the staining of a selected tissue and the stain is selected because it is attuned to absorb the energy from a given radiant energy source, rather than selecting a laser source for a particular biological substrate as is current practice. The radiant energy source is then sufficient to destroy stained tissues, which are attuned to absorb the energy from the source by the stain, through carbonization and/or vaporization. The stain enhances absorption of incoming radiant energy, which results in increased and accelerated destruction of stained tissues. The increased absorption by stained tissues then reduces over-penetration into the column of tissues underlying the stained tissue. Therefore, this method provides clinicians with the ability to selectively mark a tissue for destruction, while leaving wanted tissues generally intact. The method also allows the most efficient laser to be used on any biological substrate regardless of the wavelengths produced. For example, a stain may be applied in a liquid form directly to selected biological tissues, followed by radiating the stained area with a laser that produces a wavelength that the stain readily absorbs. The method also incorporates the use of a radiant energy opaque substance that can be applied adjacent the stained treatment area to protect against accidental or incidental exposure to healthy tissue.

In conceptual testing, a radiant energy source was selected for its ability to adjust output wattage settings nearest those used for soft tissue surgery. The 810 nm Odyssey® NAVIGATOR™ Diode laser from Ivoclar/Vivodent, Inc. was used for this study because of the variable controls and the ease of disposable tips. The laser was set to continuous mode throughout the study. The laser hand piece was mounted onto an adjustable laboratory clamp/stand in order to control the constant tip distance to the soft tissue. A steel pre-measured gauge of 1.5 mm thickness was used to ensure the tip distance was as near a consistency of 1.5 mm from the soft tissue as possible.

The soft tissue used in this study was pork loin, which was intended to closely mimic human tissue. The wattage settings used in the test were 0.1, 0.2, 0.3, 0.4, 0.5, 1, 2, and 3. A total of 5 stain groups were selected as test groups: no stain (control group) and FD&C Green #3, FD&C Blue #2, Indocyanine green, and Carbon black. A maximum of 1 minute was selected as the duration of time to determine the carbonization treatment window. The criterion to measure whether the soft tissue achieves a state of carbonization was to examine the time it takes for a gray to black dot to form immediately beneath a weak aiming beam. The study considers the formation of the usual black or gray spots as evidence of carbonization and/or combustion. The formation of a gray to black dot or spot is considered a positive test and the time of initiation is noted. The formation of no spot or dot is a negative test or none formed.

The experiment in general consisted of laying a fairly flat piece of pork loin on a flat surface and positioning the laser tip with the aid of the steel gauge to about 1.5 mm from the surface. To the pork loin was then applied a coat of the various stains and subsequently irradiated at the various power settings until carbonization was achieved or 1 minute of time elapsed. The time was controlled with a stopwatch. The following table presents the results:

| | No Stain | FD&C green | | Indocyanine | |
|---|---|---|---|---|---|
| | (control) | #3 | FD&C Blue #2 | Green (ICG) | Carbon Black |
| 0.1 | No | No | No | No | No |
| Watt | carbonization | carbonization | carbonization | carbonization | carbonization |
| 0.2 | No | No | No | No | No |
| Watt | carbonization | carbonization | carbonization | carbonization | carbonization |
| 0.3 | No | No | No | No | 58 seconds |
| Watt | carbonization | carbonization | carbonization | carbonization | |
| 0.4 | No | No | No | 47 seconds | 26 seconds |
| Watt | carbonization | carbonization | carbonization | | |
| 0.5 | No | No | No | 21 seconds | 11 seconds |
| Watt | carbonization | carbonization | carbonization | | |
| 1.0 | No | No | No | 5 seconds | 1 second |
| Watt | carbonization | carbonization | carbonization | | |
| 2.0 | No | No | No | 2 seconds | 1 second |
| Watt | carbonization | carbonization | carbonization | | |
| 3.0 | No | No | No | 1 second | 1 second |
| Watt | carbonization | carbonization | carbonization | | |

The stains were chosen for their various absorption efficiencies with respect to a λₘₐₓ of 810 nm. The absorption efficiency is merely a percentage of energy absorbed by the stained tissue with respect to energy output. Carbon black was selected as a universal stain with absorption efficiencies above 95% over a wide range of wavelengths; as can be seen from the data how effective it was over the control. Indocyanine Green was selected for its known λₘₐₓ near 810 nm and has absorption efficiency greater than about 90%; it also allowed carbonization of soft tissue at a much lower wattage than an unmatched stain and/or control groups. FD&C Blue #2 was selected for its minimal absorption characteristics at 810 nm, with only about a 30% efficiency it did no better than the control, though it would in theory initiate carbonization sooner than the control at higher wattages. FD&C green #3 was selected because it had insignificant absorption efficiency at 810 nm and as demonstrated—did no better than the control.

The data demonstrates that when the absorption characteristics of a stain are matched to the wavelength of a radiant energy source, the power output required to initiate carbonization is significantly reduced. Carbon black initiated carbonization with as little as 0.3 watts at a distance of 1.5 mm from the pork loin. On the other hand, the control did not initiate carbonization at 3.0 watts at 1.5 mm. This study shows that it is possible to paint any given tissue, regardless of the absorption characteristics of said tissue and carbonize said tissue selectively and at a much lower wattage. It also demonstrates that at these lower wattage settings, unstained tissue will be unharmed by the radiant energy.

An actual in vivo clinical test recently performed confirmed the efficacy of the present invention. In the test, a laser source emitting laser energy having a wavelength of about 810 nm and a power level of about 5 W was used to expose a cancerous tumor having a volume about 9 mm in diameter to laser energy for about 5 minutes. Necrosis of the tumor began after about 1 minute of exposure, and the tumor was substantially destroyed after about 5 minutes, resulting in destruction of all or substantially all of the cancerous cells exposed to the laser energy.

Preferred embodiments will depend upon the laser available to a clinician. However, in each case, the stain should have an absorption efficiency of greater than 90% at the given laser source's λₘₐₓ. Obviously, the higher the efficiency, the lower power output from the laser source will be necessary and less collateral damage to healthy tissue will occur. As illustrated above, for an 810 nm diode laser, carbon black or indocyanine green may be used. In the case of an absorption efficiency of 95% or greater, only 0.3 W of power may be used as a minimum. At an efficiency of 90% or greater, the power output may be 0.4 W or greater. Stronger power outputs may be used to lessen treatment time and still not affect untreated tissue as illustrated in the conceptual test. Other dyes may be used so long as they have a λₘₐₓ that allows for an absorption efficiency of 90% or greater for a given wavelength of energy. For example, toluidine blue has a λₘₐₓ at 626 nm, so it may be used with a radiant energy source capable of emitting such energy at that wavelength. Bromophenol blue has three λ-maxima, at 383, 422 and 589 nm respectively, and may be used with a corresponding radiant energy source for either of those three maxima. The actual power output should be left to the clinician to determine based on each particular case, as size and location of the tumor will also factor into treatment times and power output. It is possible for treatment times to extend as little as one minute or as long as an hour or more depending on the wattage used, size of the tumor, absorption efficiency and other factors.

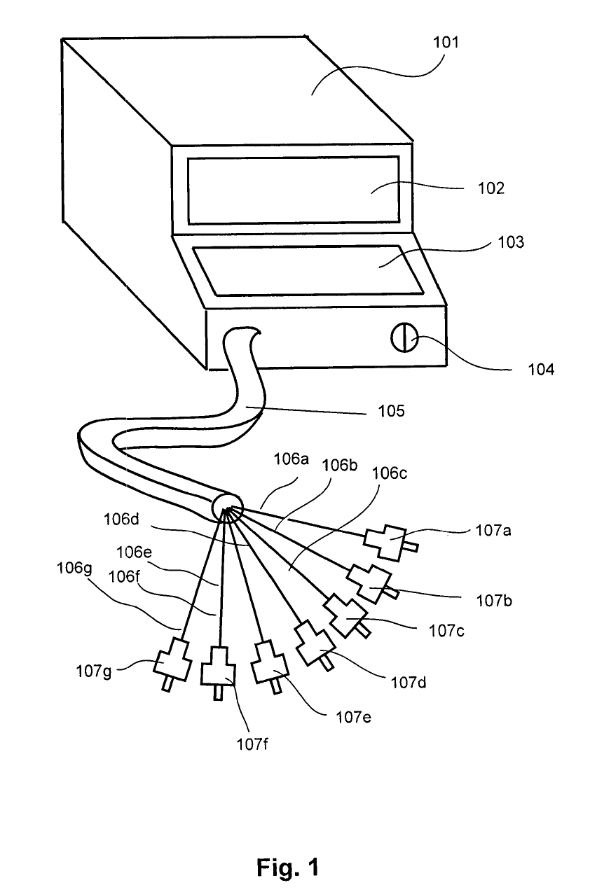

FIG. 1 depicts an example laser system 101 that can be used for tumor treatment. The laser system 101 contains a laser light source, control circuits, and other managing/control components, energy supply and circuitry. A display panel 102 displays all laser and treatment information. A control panel 103 has buttons or switches to control the laser's operation. A key switch 104 may be used to control the main electrical on/off for safety reasons. A fiber bundle cable 105 may be used to transport light out of the main laser module to some remote location for treatment use. The fiber bundle may be broken down into numerous individual fibers 106a through 106g. Each fiber may have an end connector, 107a through 107g respectively, to facilitate transmission of laser energy from the laser system 101 to a delivery device for delivering laser energy to tumor cells.

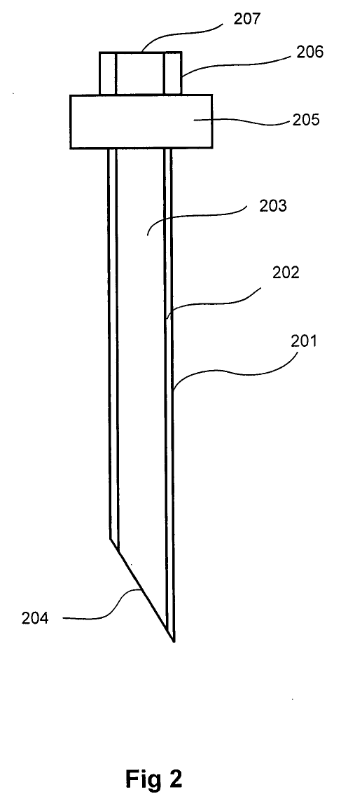

FIG. 2 depicts an example fiber needle 200 that can be used to deliver the laser energy to tumor cells. The fiber needle may include a rigid housing (such as metal or plastic) with a stem 201, a channel 202, and a fiber 203 inside the channel. The end of the needle may have a sharp point and an angled surface 204. The end of the fiber is polished to the same angle as the metal housing to create a sharp point for insertion. Laser energy is delivered through the fiber. The top side of needle includes a fiber connector 206 and an abutment 205 so that the needle 200 can connect to the fiber with the connector from the laser unit. The top side of the needle includes a polished surface 207 for connection to the connector from individual fibers of the fiber bundle mentioned above. The sharp fiber needle may be inserted into the body in any location where cancerous cells are believed to be located in order to deliver laser energy directly to those cells.

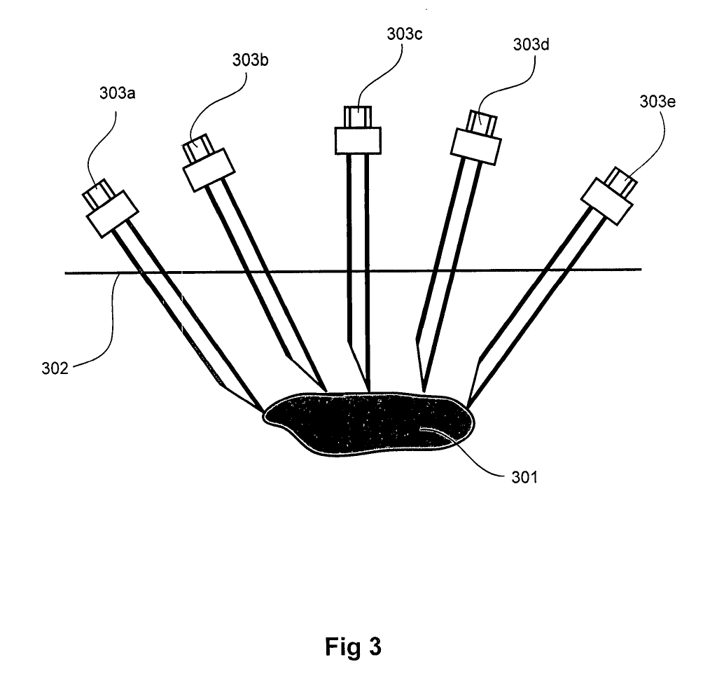

FIG. 3 depicts an example of using multiple fiber needles to deliver laser energy to tumor cells. If desired, laser energy may be delivered to tumor cells at one or more points such as depicted, or it may be delivered in a footprint covering a larger area if desired. A cancer tumor 301 in a human body below the skin surface 302 is located, and fiber needles 303a, 303b, 303c are inserted into the human body and pointed toward the tumor. It is possible to deliver the laser energy from outside the body without a needle invading the body, but it may be desirable to insert needles into unaffected tissue so that laser energy may be delivered directly to the tumor. The fiber needles may surround or partially surround the cancer tumor. The number of fiber needles to be used in treatment depends on the size and location of cancer tumor. The depth of the needle insertion depends on the location of the tumor. The length or height of the fiber needle can be different based on particular requirements of different treatment situations.

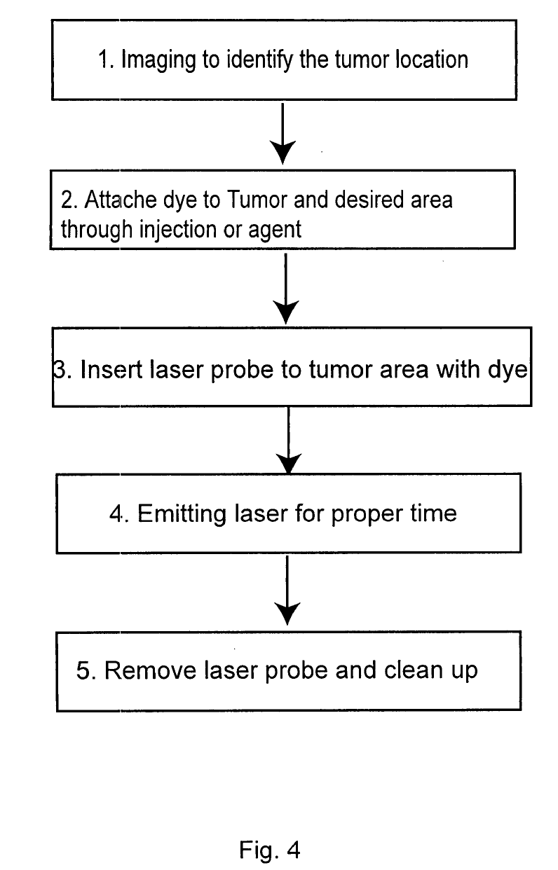

FIG. 4 illustrates the steps typically carried out in practicing the present invention arthroscopically. For example, the first step 401 typically requires that the location of a tumor or cells be identified. This step is carried out using conventional medical imaging means such as x-ray or magnetic resonance. The next step 402 is to attach a stain to the tumor. This step is typically carried out through injection or agent using one of the direct or indirect methods described above—e.g., through systemic injection of a stain or stain combined with tumor seeking compound into the bloodstream (indirect) or through non-systemic mechanical application using a syringe (direct). The third step 403 concerns placement of the fiber or fiber needles adjacent the tumor. As explained above, this step can be performed using a single fiber needle or a plurality of needles arranged advantageously about the volume of the tumor. The fourth step 404 requires operation of the laser over a specified time interval. As explained, the laser may be operated in a variety of ways, including pulsing, constant-wave or modulated fashion. The final step 405 involves removal of the fiber needle or needles following irradiation of the tumor.

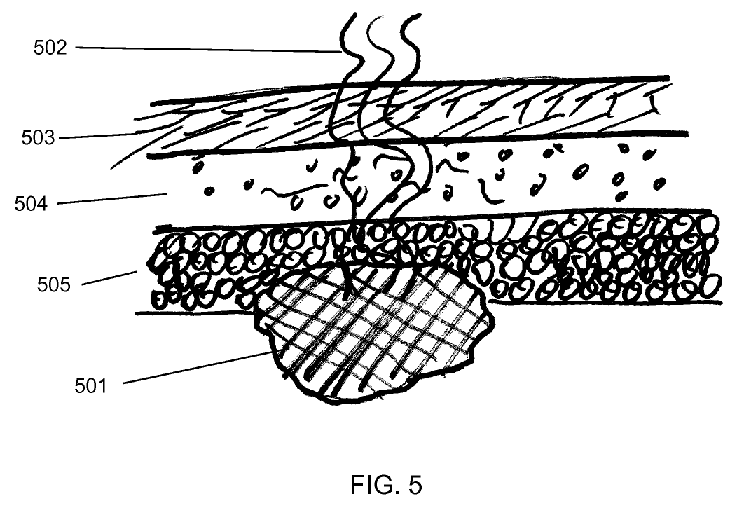

Another embodiment of this invention, shown in FIG. 5, allows the use of radiant energy at wavelengths to which soft tissue of the human body is normally transparent. Various wavelengths are known to which living tissue is transparent, X-rays and gamma ray are such examples. However, radiant energy with a wavelength between 900 and 1100 nm would also pass though living soft tissue normally without causing damage to said tissue. It is possible to stain a tumor 501 with stain that absorbs such energies and follow the methods taught herein for destruction of said tumor. The tumor would then be capable of absorbing the radiant energy 502 while residing underneath a column of layers of tissue 503, 504, 505 transparent to that same energy. In such a treatment, no entry into the living body is necessary as unstained soft tissue would be transparent to the energy as radiant energy will be directed towards the tumor and pass through the “transparent” tissue with no harm to that tissue. The tumor, however, being stained with the appropriate non-transparent stain, will absorb the radiant energy and be destroyed according to the earlier teachings of this invention. Appropriate materials for such “stains” include: amminium dyes as for example metal tris amminium dyes or metal tretrakis amminium dyes wherein the metal includes boron, iron, cobalt, nickel, copper, or zinc such as cobalt tris amminium various metal dithiolene dyes wherein the metal includes boron, iron, cobalt, nickel, copper, or zinc, such as nickel dithiolene, and the like; various diphenylmethane, triphenylmethane and related dyes; various quinone dyes such as naphthoquinone dyes; various azo type dyes; various benzene dithiol type metal complex dyes wherein the metal includes boron, iron, cobalt, nickel, copper, or zinc; various pyrylium type dyes; various squarylium type dyes; various croconium type dyes; various azulenium type dyes; various dithiol metal complex type dyes; various indophenol type dyes; and various azine type dyes. Exposing hard tissue, such as bone, to such radiant energy should be avoided.

While compositions and methods have been described and illustrated in conjunction with a number of specific ingredients, materials and configurations herein, those skilled in the art will appreciate that variation and modifications may be made without departing from the principles herein illustrated, described, and claimed. The present invention, as defined by the appended claims, may be embodied in other specific forms without departing from its spirit or essential characteristics. The configurations described herein are to be considered in all respects as only illustrative, and not restrictive. All changes which come within the meaning and range of equivalency of the claims are to be embraced within their scope.

We claim:

1. A method for treating tumor cells within a living body using a laser system through which laser light may be emitted comprising the steps of:
a. identifying the location of a tumor;
b. selectively staining the tumor with a stain such that non-targeted tissue is left substantially unstained; and
c. communicating radiant energy to the tumor with sufficient energy to destroy the tumor through at least one process of destruction selected from the group of processes of destruction consisting of carbonization and vaporization, while simultaneously minimizing harm to tissue surrounding the tumor.

2. The method of claim 1, wherein the stain has an absorption efficiency to the radiation energy higher than 80%.

3. The method of claim 1, the radiant energy emitted having a wavelength in the range from about 200 nm to about 8,000 nm.

4. The method of claim 1, wherein the laser system operating at a power level of at least 0.3 Watts.

5. The method of claim 1, wherein the laser system includes a plurality of fibers, each capable of directing at least a portion of the radiant energy communicated to the tumor.

6. The method of claim 1, wherein the stain is selected from the group consisting of indocyanine green, carbon black, FD&C Blue #2, nigrosin, FD&C black shade, FD&C blue #1, methylene blue, FD&C blue #2, malachite green, D&C green #8, D&C green #6, D&C green #5, ethyl violet, methyl violet, FD&C green #3, FD&C red #3, FD&C red #40, D&C yellow #8, D&C yellow #10, D&C yellow # 11, FD&C yellow #5, FD&C yellow #6, neutral red, safranine O, FD&C carmine, rhodamine G, napthol blue black, D&C orange #4, thymol blue, aurarnine O, D&C red #22, D&C red #6, xylenol blue, chrysoidine Y, D&C red #4, sudan black B, D&C violet #2, D&C red #33, cresol red, fluorescein, fluorescein isothiocyanate, bromophenol red, D&C red #28, D&C red #17, amaranth, methyl salicylate, eosin Y, lucifer yellow, thymol, and dibutyl phthalate.

7. The method of claim 1, wherein the laser system is selected from the group consisting of semiconductor lasers, solid state lasers, and gas lasers.

8. The method of claim 1, wherein the laser system emits radiant energy of a modulating power level in the range of from 0.1 watt to 30 watts.

9. The method of claim 1, wherein the tumor or cells are exposed to the laser light for a time duration that is within the range of from about 1 second to about 1 hour.

10. The method of claim 1, wherein the step of locating a region within the body that contains a tumor is performed using one of the methods in the group consisting of three-dimensional imaging, laser scanning, magnetic resonance imaging, x-ray imaging, and CT scanning.

11. The method of claim 1, wherein the step of identifying the location of a tumor includes systemic injection of a stain into the bloodstream.

12. The method of claim 1, wherein the step of identifying the location of a tumor includes systemic injection of a stain combined with a tumor seeking compounds into the bloodstream.

13. The method of claim 10, wherein the same stain is used in the steps of identifying the location of the tumor and staining the tumor.

14. The method of claim 1, wherein the step of staining the tumor includes direct application of the stain using a syringe.

15. The method of claim 1, wherein the step of identifying the location of the tumor includes systemic injection of a chemical imaging solution.

16. The method of claim 15, wherein the chemical imaging solution also comprises a stain.

17. The method of claim 1, the laser system emitting radiant energy at a wavelength to which living tissue is transparent and the radiant energy passes through a column of unstained tissue to reach the tumor.

18. The method of claim 17, the wavelength of the radiant energy being within the range between 900 and 1100 nm, inclusively.

19. The method of claim 18, the stain is selected from the group consisting of amminium dyes, metal tris amminium dyes, metal tretrakis amminium dyes, metal dithiolene dyes, benzene dithiol type metal complex dyes, wherein the metal includes boron, iron, cobalt, nickel, copper, or zinc; diphenylmethane; triphenylmethane; quinone dyes; azo type dyes; pyrylium type dyes; squarylium type dyes; croconium type dyes; azulenium type dyes; dithiol metal complex type dyes; indophenol type dyes; and azine type dyes.

20. A method of tumor treatment comprising a laser system having a fiber extending through a needle configured for insertion into the said body through which laser light may be emitted, the method further comprising the steps of:
a. introducing a stain material into a living body, such that tumor cells will preferentially absorb the stain;
b. after locating the tumor cells, inserting the fiber needle into the human body so that the end of the fiber needle is in close proximity to the tumor cells and so that the fiber needle tends to point in the direction of the tumor cells; and
c. causing emission of laser light from the laser system, through the fiber, through the fiber needle and thence to the tumor cells for the destruction thereof; and
wherein the stain is selected because it has an absorption efficiency of energy at a given λₘₐₓ of the laser light that is greater than the absorption efficiency of healthy tissue surrounding the tumor cells.

21. The method of claim 20, wherein the biological stain is selected from the group consisting of indocyanine green, carbon black, FD&C Blue #2, nigrosin, FD&C black shade, FD&C blue #1, methylene blue, FD&C blue #2, malachite green, D&C green #8, D&C green #6, D&C green #5, ethyl violet, methyl violet, FD&C green #3, FD&C red #3, FD&C red #40, D&C yellow #8, D&C yellow #10, D&C yellow # 11, FD&C yellow #5, FD&C yellow #6, neutral red, safranine O, FD&C carmine, rhodamine G, napthol blue black, D&C orange 774, thymol blue, auramine O, D&C red #22, D&C red #6, xylenol blue, chrysoidine Y, D&C red #4, sudan black B, D&C violet #2, D&C red #33, cresol red, fluorescein, fluorescein isothiocyanate, bromophenol red, D&C red #28, D&C red #17, amaranth, methyl salicylate, eosin Y, lucifer yellow, thymol, and dibutyl phthalate.

22. The method of claim 21, the radiant energy having a wavelength of about 810 nm and the biological stain being selected from the group of biological stains consisting of carbon black and indocyanine green.

23. The method of claim 20, wherein the energy emitted from the laser has a wavelength in the range from about 200 nm to about 8,000 nm.

24. The method of claim 20, wherein the laser operates at a power level of at least 0.3 Watts.

25. A method treating tumor cells residing proximate at least one layer of healthy tissue, the method comprising:
a. selecting a radiant energy source capable of emitting radiant energy at a wavelength that is substantially transparent to the at least one layer of healthy tissue;
b. selectively staining tumor cells with a dye that has an absorption maxima nearest said wavelength, wherein cells in the at least one layer of healthy tissue remain unstained; and
c. radiating a treatment area, the tumor cells and a column of healthy tissue between the tumor and a tissue surface defining the treatment area, with the radiant energy until the stained cells are destroyed;
wherein the radiant energy is minimally absorbed and passes through the healthy tissue.
